(19) United States
(12) Patent Application Publication
(10) Pub. No.: US 2026/0294231 A1
(43) Pub. Date: Oct. 1, 2026

(54) VISION TESTING DEVICE

(71) Applicant: FIM MEDICAL, VILLEURBANNE (FR)

(72) Inventor: Gilbert ROZET, Saint-Pierre-de-Chandieu (FR)

(21) Appl. No.: 19/461,180

(22) Filed: Jan. 27, 2026

(30) Foreign Application Priority Data
Jan. 30, 2025 (FR) 2500976

(51) Int. Cl. A61B 3/00; A61B 3/032

(52) U.S. Cl. CPC A61B 3/005; A61B 3/032

(57) ABSTRACT

A patient vision testing device designed to occupy a first configuration in which a first optical path is provided between an observation position of one eye of the patient and an image display device and including a movable member having at least two movable member housings intended to alternately occupy a housing position on the first optical path (30, 130, 230) by rotation of the movable member about a movable member rotation axis substantially coplanar and parallel to one direction of a portion of the first optical path extending between the observation position of the patient's eye and the housing position.

### CROSS-REFERENCE TO RELATED APPLICATIONS

This application claims the benefit of French patent application no. 2500976, filed on 30 Jan. 2025, the disclosure of which is incorporated herein by reference in its entirety.

### TECHNICAL FIELD

This disclosure, which falls within the field of ophthalmic devices, concerns in particular a vision testing device allowing to screen for potential visual defects in a patient and/or to perform a diagnosis relating to the patient's vision.

### BACKGROUND

A comprehensive screening and diagnosis of a patient's visual defects necessarily involve performing various tests for distance, near, and intermediate vision.

Whether for near or distance vision, national legislation in different countries generally requires tests to be performed by simulating different distances.

For example, in occupational health testing, the simulated distance for near vision testing can range from 25 to 40 centimeters, including 33 centimeters, depending on the country.

However, existing devices allowing to test both near and distance vision offer only a limited number of simulated distances, which hinders their international commercialization.

In some contexts, it is also sometimes necessary to use several devices to fully test a single and same patient, which is particularly cumbersome and entails additional costs for the practitioner.

There is therefore a need for a more adaptable vision testing device, that is to say that can in particular simulate a wider range of distances.

The current vision testing devices are also generally bulky, making them difficult to transport and store, and it is further common for the patient to have to operate the device themselves while being screened.

There is therefore a need for a vision testing device that is both more compact and easier to use, that is to say requiring no special handling from the patient being tested.

### SUMMARY

The present disclosure aims to overcome all or part of the aforementioned drawbacks.

To this end, the present disclosure provides a patient vision testing device that is more compact, easier to use, and modular than existing devices, and which makes it possible for comprehensive screening of visual defects in the patient and/or precise diagnostic procedures for the patient's vision.

According to a first aspect, the present disclosure relates to a patient vision testing device capable of occupying at least one first configuration in which a first optical path is provided between an observation position of a patient's eye and an image display device, the vision testing device comprising a movable member having at least two movable member housings intended to occupy alternately on the first optical path a housing position by rotation of the movable member about a movable member axis of rotation substantially coplanar with and parallel to a direction of a portion of the first optical path extending between the observation position of the patient's eye and the housing position.

According to one embodiment, the vision testing device further comprises a primary optical element of the first optical path disposed on the first optical path.

According to one possibility, the primary optical element of the first optical path is disposed between the observation position of the patient's eye and the housing position.

According to another possibility, the primary optical element of the first optical path is disposed between the housing position and the image display device. According to this possibility, the housing position is located between the observation position of the patient's eye and the primary optical element of the first optical path.

According to one embodiment, each of said at least two movable member housings permanently integrates a secondary optical element of the first optical path, or each of said at least two movable member housings is capable of temporarily receiving a secondary optical element of the first optical path.

According to one possibility, the vision testing device may comprise, on the first optical path, a secondary optical element of the first optical path and be devoid of a primary optical element of the first optical path.

According to another possibility, the vision testing device may comprise, on the first optical path, a primary optical element of the first optical path as well as a secondary optical element of the first optical path.

According to one embodiment, the primary optical element of the first optical path is a primary lens of the first optical path.

According to one embodiment, the secondary optical element of the first optical path is a secondary lens of the first optical path or a prism of the first optical path.

According to one embodiment, the vision testing device is further capable of occupying a second configuration in which a second optical path is provided between the observation position of the patient's eye and the image display device, the vision testing device further comprising an image return system comprising a first mirror and a second mirror.

According to one embodiment, the image return system is at least partially movable to allow the vision testing device to be switched from the first configuration to the second configuration and vice versa.

According to one embodiment, the second mirror is movable between a deployed second-mirror position occupied by the second mirror when the vision testing device occupies the first configuration, and a retracted second-mirror position occupied by the second mirror when the vision testing device occupies the second configuration.

According to one embodiment, the vision testing device comprises at least one optical element of the second optical path arranged on the second optical path.

According to one embodiment, the at least one optical element of the second optical path comprises a prism of the second optical path and/or a second optical path lens.

According to one embodiment, the image return system comprises a third mirror.

According to one embodiment, when the vision testing device occupies the first configuration, an additional first optical path is provided between an observation position of the patient's other eye and the image display device, the vision testing device comprises an additional movable member comprising at least two additional movable member housings intended to alternately occupy an additional housing position on the additional first optical path by rotation of the additional movable member about an additional movable member axis of rotation that is substantially coplanar with and parallel to a direction of a portion of the additional first optical path extending between the observation position of the patient's other eye and the additional housing position.

According to one possibility, the first optical path corresponds to a distance vision optical path and said first configuration corresponds to a distance vision configuration, while the second optical path corresponds to a near vision optical path and said second configuration corresponds to a near vision configuration.

According to this latter possibility, the vision testing device has the advantage of allowing a patient, from a single observation position, to view images displayed on an image display device via both a distance vision optical path and a near vision optical path, without any risk of confusion for the patient.

Indeed, the image return system is adapted to return the images displayed on the image display device toward the observation position of the patient's eye and the returned images reach the patient's eye via the distance vision optical path (or first optical path) when the vision testing device occupies the distance vision configuration (or first configuration), and via the near vision optical path (or second optical path) when the vision testing device occupies the near vision configuration (or second configuration).

According to one possibility, the movable member has at least five movable member housings, and each of these five movable member housings is adapted to receive an optical element, such as for example a lens.

It is specifically the configuration of the movable member, with an axis of rotation coplanar with and parallel to the portion of the first optical path extending between the observation position of the patient's eye and the housing position, that makes it possible for the arrangement of multiple movable member housings while offering increased compactness for the vision testing device.

Thanks to these features, the vision testing device is capable of simulating, particularly in the first configuration, a greater number of test distances than existing devices.

Thus, the vision testing device is more modular than known devices, without its structure being more cumbersome or its manufacturing more complex.

Due to its rotational mobility about the movable member axis of rotation, the movable member is adapted to occupy the housing position in turn for each of the five movable member housings.

According to one possibility, the vision testing device comprises means for defining the observation position of the patient's eye.

According to this possibility, the means for defining the observation position of the patient's eye comprise a system for supporting, positioning, and/or maintaining the patient's head in position, such as a headrest or chin rest, or even eye-tracking glasses.

According to one possibility, the vision testing device comprises an image display device making it possible, for example, to display optotypes or other symbols observable by the patient's eye from the observation position of the patient's eye.

According to this possibility, the image display device is integrated into the vision testing device.

According to another possibility, the vision testing device is designed to operate with a remote image display device.

According to this possibility, the vision testing device does not integrate an image display device.

According to one possibility, the vision testing device is designed to operate with an additional image display device, in addition to the image display device.

According to this possibility, the additional image display device can be identical to the image display device.

According to one possibility, the primary lens of the first optical path is mounted on a primary lens support of the first optical path, said support being immovable.

In particular, the primary lens of the first optical path is intended to cooperate alternately with each of the at least five movable member housings, each receiving (or not) for example, a separate secondary lens of the first optical path, so as to form different optical arrangements.

According to one possibility, the movable member takes the form of a circular barrel, and in particular that of a disc with a small thickness.

According to one possibility, the movable member has five movable member housings, to receive optical elements, regularly distributed over the surface of the movable member, each of the five movable member housings thus being adapted to receive a secondary lens of the first optical path.

According to another possibility, the movable member has six movable member housings, for receiving optical elements, which are regularly distributed over the surface of the movable member.

According to one possibility, the vision testing device comprises a casing intended to accommodate and protect at least some of the elements of the vision testing device, the image display device being, for example, provided on one of the faces of the casing, the observation position of the patient's eye being located outside said casing.

According to one possibility, at least two openings are provided in the casing, respectively to define the first optical path and to define the second optical path.

Particularly, at least one first optical path opening is provided to define the first optical path, and at least one second optical path opening is provided to define the second optical path.

According to one possibility, the primary lens of the first optical path is arranged on the first optical path between the observation position of the patient's eye and the housing position.

In the embodiment in which each of the at least five movable member housings permanently integrates a secondary lens of the first optical path, a secondary lens of the first optical path is permanently fixed within each of the at least five movable member housings and cannot be removed or replaced.

In this embodiment, the movable member can advantageously be manufactured as a single unit with integrated lenses, for example, by thermoplastic injection molding, which is cost-effective.

Alternatively, each of the secondary lenses of the first optical path can respectively be glued into each of the at least five movable member housings.

The embodiment in which each of said at least five movable member housings is adapted to temporarily receive a secondary lens of the first optical path is advantageous because it makes it possible to interchange or replace the secondary lenses of the first optical path, thus increasing the modularity of the vision testing device, and also because it makes it possible for an optical arrangement in which the primary lens of the first optical path is not associated with any secondary lens of the first optical path.

According to this embodiment, the movable member has, at each of the housings, a temporary fixing system for an optical element.

In this embodiment, each lens can, for example, be snap-fitted, clipped, or screwed, respectively, into a movable member housing.

According to one possibility, the image return system is such that the second mirror is movable, and, for example, rotatable.

According to one possibility, the first mirror is immovable, and in particular mounted on an immovable first mirror support.

According to a possibility, the second mirror is mounted on a second-mirror support rotatable about an axis of rotation of the second-mirror support, the second-mirror support is adapted to move the second mirror from the deployed second-mirror position to the retracted second-mirror position and vice versa.

According to one possibility, the second-mirror support is motorized, and the vision testing device comprises a second-mirror support actuating device and a second-mirror support drive device comprising at least one motor.

According to one possibility, when it occupies the deployed second-mirror position, the second mirror obstructs the second optical path.

According to one possibility, when it occupies the retracted second-mirror position, the second mirror clears the second optical path, as well as the first optical path is interrupted.

According to one possibility, the second optical path prism is arranged on the second optical path between the image display device and the patient's eye position.

According to one possibility, the second optical path prism, mounted in a removable way on a prism support of the second optical path, is interchangeable. According to this possibility, different second optical path prisms can be used with the vision testing device.

In an advantageous possibility, the vision testing device comprises both a second optical path prism and a second optical path lens arranged on the second optical path, allowing for a specific optical arrangement.

According to one possibility, the second optical path lens is arranged on the second optical path between the image display device and the second optical path prism.

According to one possibility, the second optical path lens, mounted in a removable way on a second optical path lens support, is interchangeable.

This last possibility is advantageous because it makes it possible for different optical arrangements, that is to say, different prism-lens combinations, without having to change the second optical path prism.

According to one possibility, the third mirror is arranged on the first optical path between the image display device and the second mirror when the second mirror occupies the deployed second-mirror position, that is to say, when the vision testing device occupies the first configuration.

Furthermore, when the second mirror occupies the retracted second-mirror position, that is to say, when the vision testing device occupies the second configuration:
The third mirror is arranged on the second optical path between the image display device and the second optical path prism when the vision testing device does not comprise a second optical path lens;
The third mirror is arranged on the second optical path between the image display device and the second optical path lens when the vision testing device comprises a second optical path lens.

According to one possibility, the third mirror is immovable, that is to say, mounted, for example, on a third-mirror support, said support being immovable.

According to some of the embodiments described above, the vision testing device has the advantage of being able to test the patient's vision globally, and thus, for example, to simultaneously screen any visual defects affecting one or both of the patient's eyes.

Thus and according to one possibility, the vision testing device comprises a primary lens of the additional first optical path arranged on the additional first optical path and for example located between the observation position of the patient's other eye and the additional housing position.

According to one possibility, the primary lens of the additional first optical path is identical to the primary lens of the first optical path.

According to one possibility, when the vision testing device occupies the second configuration, an additional second optical path is provided between the observation position of the patient's other eye and the image display device.

The image return system is also adapted to return the images displayed on the image display device to the observation position of the patient's other eye, and the returned images reach the patient's other eye via the additional first optical path when the vision testing device occupies the first configuration, and via the additional second optical path when the vision testing device occupies the second configuration.

According to one possibility, the vision testing device comprises an additional second optical path prism arranged on the additional second optical path.

According to one possibility, the additional second optical path prism is identical to the second optical path prism.

According to one possibility, the additional second optical path prism is arranged on the additional second optical path between the image display device and the patient's other eye position.

According to one possibility, the vision testing device comprises an additional second optical path lens arranged on the additional second optical path.

In yet another possibility, the vision testing device comprises both an additional second optical path prism and an additional second optical path lens arranged on the additional second optical path.

According to this possibility, the additional second optical path lens is, for example, arranged on the additional second optical path between the image display device and the additional second optical path prism.

According to one embodiment, the movable member and the additional movable member are configured to be rotatable in a synchronized manner.

According to this embodiment, a rotational movement of the movable member (respectively the additional movable member) drives in rotation the additional movable member (respectively the movable member) in a synchronized manner. Thus, when the movable member is displaced in rotation at a given speed, the movable member is also displaced in rotation at the same speed, but in the opposite direction.

This embodiment is advantageous because it eliminates the need for any additional manipulation of the vision testing device by an operator or the patient.

The synchronized rotational mobility of the movable member and the additional movable member is obtained by means of a gearing mechanism, a first part of the gearing mechanism is provided on the movable member, and a second part of the gearing mechanism, complementary to the first part of the gearing mechanism, is provided on the additional movable member.

For example, the first part of the gearing mechanism comprises a first set of teeth, for example, arranged at the periphery of the movable member, and the second part of the gearing mechanism comprises a second set of teeth, complementary to the first set of teeth, and, for example, arranged at the periphery of the additional movable member.

The vision testing device may further comprise means for actuating and means for driving in rotation the movable member and/or the additional movable member.

According to one embodiment, the vision testing device comprises a drive mechanism capable of simultaneously driving in rotation the movable member and the additional movable member, said drive mechanism comprising, for example, a motor.

According to this embodiment, the synchronized rotational movement of the movable member and the additional movable member is obtained by means of the drive mechanism, and when the movable member is displaced in rotation at a given speed, the additional movable member is also displaced in rotation at the same speed.

According to one embodiment, the drive mechanism comprises a first drive mechanism part provided on the movable member, a second drive mechanism part provided on the additional movable member, and a third drive mechanism part comprising a rotatable pinion.

According to this embodiment, the third drive mechanism part is complementary to both the first drive mechanism part and the second drive mechanism part.

For example, the first drive mechanism part comprises a first set of teeth arranged at the periphery of the movable member, the second drive mechanism part comprises a second set of teeth arranged at the periphery of the additional movable member, and the pinion comprises a third set of teeth complementary to both the first set of teeth and the second set of teeth.

Thanks to this arrangement, and in particular to the rotatable pinion, the direction of rotation of the additional movable member is identical to the direction of rotation of the movable member. In addition, the additional movable member can, for example, be exactly identical to the movable member.

According to one possibility, the pinion has a thin disc shape and comprises the third set of teeth at its periphery.

According to one possibility, the pinion is motorized, and the vision testing device therefore comprises a device for actuating the pinion and a device for driving the pinion comprising at least one motor, which advantageously further reduces the handling required by the user or an operator on the vision testing device.

According to another possibility, the movable member and the additional movable member are configured to be rotatable independently of each other, and their respective movements are desynchronized.

According to a second aspect, the present disclosure relates to a method for using the previously described patient vision testing device, said use being intended to screen at least one visual defect affecting one or both of the patient's eyes, and/or to make a diagnosis concerning the patient's vision.

The method for using the vision testing device may comprise the following steps:

A patient positions themselves in a condition of use of the vision testing device, such that a patient's eye is arranged at the observation position of the patient's eye, and such that the patient's other eye is positioned at the observation position of the patient's other eye.

The vision testing device is brought to occupy the second configuration.

The image display device displays a first image, and the patient attempts to decipher this first image, which is displayed by the image display device and returned by the image return system of the vision testing device via the second optical path.

The vision testing device is brought to occupy the first configuration.

The image display device displays a second image, and the patient attempts to decipher this second image, which is displayed by the image display device and returned by the image return system of the vision testing device via the first optical path.

The movable member and the additional movable member are driven in rotation, and the image display device displays a third image, and the patient then attempts to decipher this third image, which is displayed by the image display device and returned by the image return system of the vision testing device via the first optical path.

The various non-incompatible aspects defined above can be combined.

### BRIEF DESCRIPTION OF THE FIGURES

The disclosure will be better understood upon reading the detailed description provided below with reference to the figures, among which:

FIG. 1 shows, in a perspective view, a patient vision testing device according to a first embodiment of the disclosure.

FIG. 2 shows, in a cross-sectional and side views, the vision testing device of FIG. 1 in a first configuration.

FIG. 3 shows, in the same cross-sectional and side views as FIG. 2, the vision testing device of FIG. 1 in a second configuration.

FIG. 4 shows, in a perspective view, a part of the vision testing device of FIG. 1.

FIG. 5 shows, in a perspective view, a variant of the part of the vision testing device shown in FIG. 4.

FIG. 6 shows, in a front view, a vision testing device according to a second embodiment of the disclosure.

FIG. 7 shows, in a cross-sectional and side view along section plane AA, the vision testing device of FIG. 6 in a first configuration.

FIG. 8 shows, in cross-sectional and side view along section plane AA, the vision testing device of FIG. 6 in a second configuration.

FIG. 9 shows, in partial perspective view, a patient vision testing device according to a third embodiment of the disclosure.

FIG. 10 shows the vision testing device of FIG. 9 in a longitudinal cross-sectional view.

### DETAILED DESCRIPTION OF THE FIGURES

The vision testing device 10 makes it possible to test a patient's vision, and in particular to detect visual defects affecting one or the other of the patient's eyes and to carry out a substantial diagnosis of the patient's vision.

The vision testing device 10 for a patient comprises a casing 11 of substantially parallelepiped shape and intended to accommodate and protect at least some of the elements of the vision testing device 10.

The vision testing device 10 comprises means (not shown) for defining an observation position of a patient's eye 50 and an observation position of the patient's other eye 60. For example, said defining means may comprise a system for supporting and maintaining the patient's head in position, such as a headrest or chin rest.

The vision testing device 10 comprises an image display device 70 making it possible, for example, to display optotypes or other symbols observable by the patient's eye 50 and the patient's other eye 60 from, respectively, the observation position of the patient's eye 50 and the observation position of the patient's other eye 60.

The image display device 70 is integrated into the vision testing device 10 and is provided on one of the faces of the casing 11.

The observation position of the patient's eye 50 and the observation position of the patient's other eye 60 are respectively located outside the casing 11.

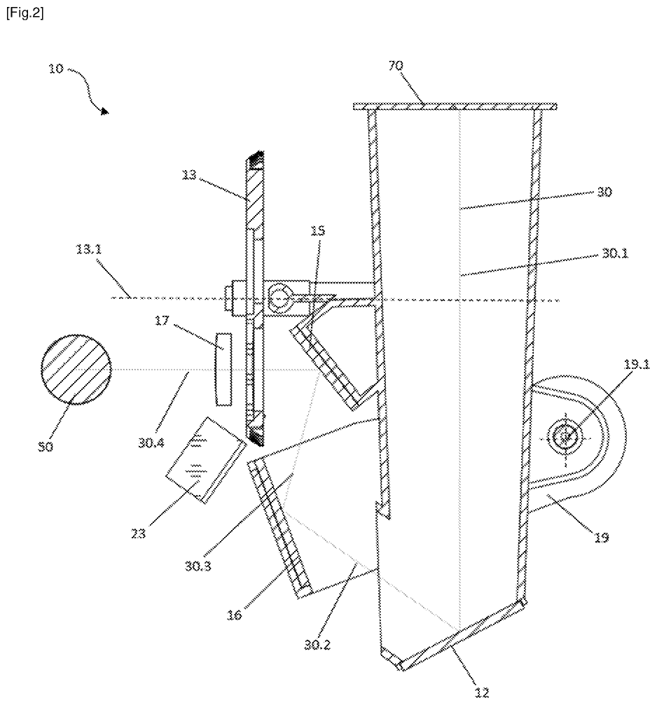

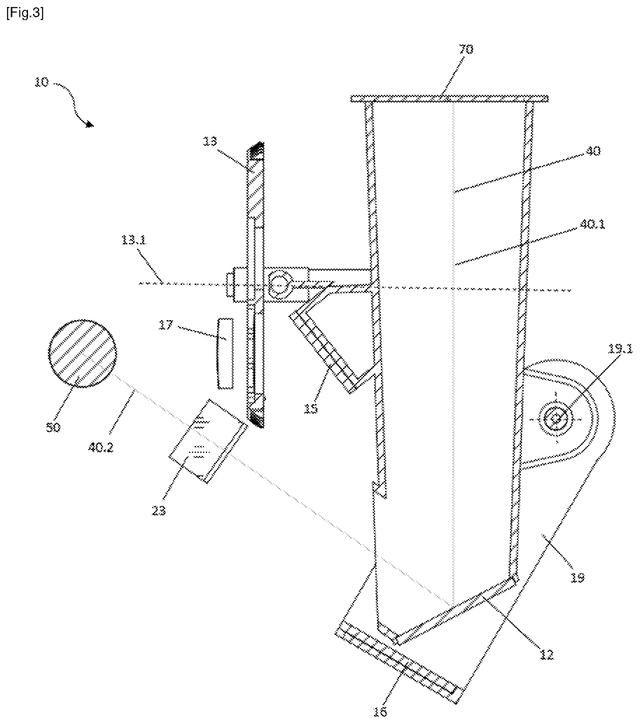

The vision testing device 10 is designed to be able to occupy:
A first configuration, or distance vision configuration, illustrated in FIG. 2, in which a first optical path 30, or distance vision optical path, is defined between the observation position of the patient's eye 50 and the image display device 70, and in which an additional first optical path (not shown in the figures), or additional distance vision optical path, is defined between the observation position of the patient's other eye 60 and the image display device 70;
A second configuration, or near vision configuration, illustrated in FIG. 3, in which a second optical path 40, or near vision optical path, is defined between the observation position of the patient's eye 50 and the image display device 70, and in which an additional second optical path (not shown in the figures), or additional near vision optical path, is defined between the observation position of the patient's other eye 60 and the image display device 70.

In order to define, respectively, the second optical path 40 and the additional second optical path, the casing 11 has a second optical path opening 21 and an additional second optical path opening 22.

In order to define, respectively, the first optical path 30 and the additional first optical path, the casing 11 also has a first optical path opening and an additional first optical path opening, which are not visible in the figures.

The vision testing device 10 comprises an image return system adapted to return the images displayed on the image display device 70 respectively toward the observation position of the patient's eye 50 and the observation position of the patient's other eye 60.

More particularly:
When the vision testing device 10 occupies the first configuration, the images returned from the image display device 70 respectively travel along the first optical path 30 to reach the patient's eye 50, and along the additional first optical path to reach the patient's other eye 60;
When the vision testing device 10 occupies the second configuration, the images returned from the image display device 70 respectively travel along the second optical path 40 to reach the patient's eye 50, and the additional second optical path to reach the patient's other eye 60.

The image return system of the vision testing device 10 comprises, in particular, a first mirror 15 and a second mirror 16.

The vision testing device 10 also comprises a primary lens of the first optical path 17 and a primary lens of the additional first optical path 18, which are arranged to be respectively positioned on the first optical path 30 facing the observation position of the patient's eye 50, and on the additional first optical path facing the observation position of the patient's other eye 60. The vision testing device 10 further comprises a movable member 13 having five movable member housings 13.2 intended to alternately occupy, on the first optical path 30, a housing position by rotation of the movable member 13 about a movable member axis of rotation 13.1 that is substantially coplanar with and parallel to a direction of a portion of the first optical path extending between the observation position of the patient's eye 50 and the housing position.

The five movable member housings 13.2 are identical to one another, each being respectively adapted to receive an optical element, such as for example a lens.

The vision testing device 10 also comprises an additional movable member 14 having five additional movable member housings 14.2 intended to alternately occupy, on the additional first optical path, an additional housing position by rotation of the additional movable member 14 about an additional movable member axis of rotation 14.1 that is substantially coplanar with and parallel to a direction of a portion of the additional first optical path extending between the observation position of the patient's other eye 60 and the additional housing position.

The primary lens of the first optical path 17 is arranged on the first optical path 30 between the observation position of the patient's eye 50 and the housing position, and the primary lens of the additional first optical path 18 is positioned on the additional first optical path between the observation position of the patient's other eye 60 and the additional housing position.

The five additional movable member housings 14.2 are identical to one another and are also identical to the five movable member housings 13.2, each of the five additional movable member housings 14.2 being respectively adapted to receive an optical element, such as for example a lens.

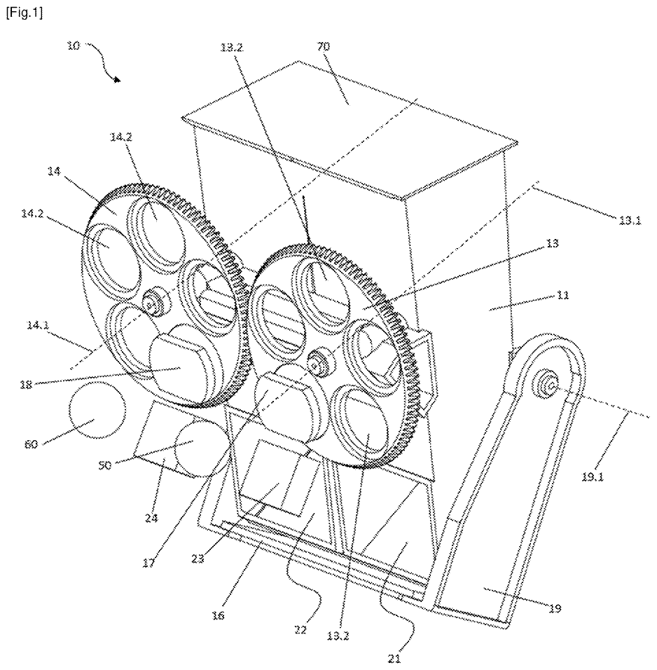

The five movable member housings 13.2 are shown in FIG. 1 without optical elements.

The five additional movable member housings 14.2 are shown in FIG. 1 without optical elements.

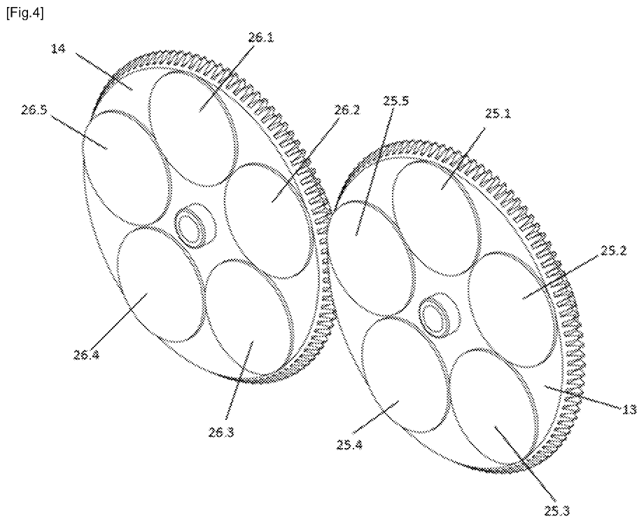

The movable member 13 and the additional movable member 14, which are therefore respectively shown in FIG. 1 as being completely devoid of optical elements, are also shown in FIG. 4 as respectively provided with five secondary lenses of the first optical path 25.1, 25.2, 25.3, 25.4, 25.5 and five secondary lenses of the first optical path 26.1, 26.2, 26.3, 26.4, 26.5.

The five secondary lenses of the first optical path 25.1, 25.2, 25.3, 25.4, 25.5 are respectively bonded in each of the movable member housings 13.2, whereas the five secondary lenses of the additional first optical path 26.1, 26.2, 26.3, 26.4, 26.5 are respectively bonded in each of the additional movable member housings 14.2.

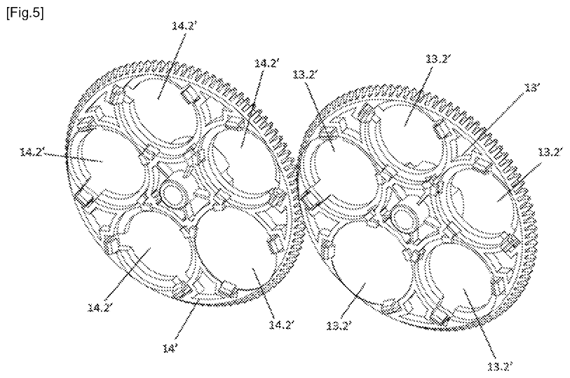

The movable member 13′ and the additional movable member 14′ shown in FIG. 5 are, respectively, a variant of the movable member 13 and of the additional movable member 14, and respectively comprise movable member housings 13.2′ and additional movable member housings 14.2′ allowing to temporary receive secondary lenses of the first optical path and secondary lenses of the additional first optical path, respectively.

Indeed, each of the movable member housings 13.2′ and of the additional movable member housings 14.2′ are identical to the others and comprise a temporary lens fastening system allowing lenses to be snap-fitted.

The movable member 13, 13′ and the additional movable member 14, 14′ are configured with respect to one another to be movable in rotation in a synchronized manner. In other words, a rotational movement of the movable member 13, 13′ (respectively of the additional movable member 14, 14′) drives in rotation, in a synchronized manner, the additional movable member 14, 14′ (respectively of the movable member 13, 13′).

Thus, when the movable member 13, 13′ is displaced in rotation at a given speed, the additional movable member 14, 14′ is also displaced in rotation at the same speed, but in the opposite direction.

The synchronized rotational mobility of the movable member 13, 13′ and of the additional movable member 14, 14′ is obtained by means of a gearing mechanism, a first part of the gearing mechanism being provided on the movable member 13, 13′, and a second part of the gearing mechanism, complementary to the first part of the gearing mechanism, being provided on the additional movable member 14, 14′.

Specifically, the first part of the gearing mechanism comprises a first set of teeth arranged at the periphery, and in particular on the circumference, of the movable member 13, 13′, and the second part of the gearing mechanism comprises a second set of teeth, complementary to said first set of teeth, arranged at the periphery, and in particular on the circumference, of the additional movable member 14, 14′.

The vision testing device 10 further comprises means for actuating and means for driving in rotation the movable member 13, 13′ and/or the additional movable member 14, 14′.

The image return system of the vision testing device 10 is partially movable so as to switch the vision testing device 10 from the first configuration to the second configuration, and vice versa.

The first mirror 15 of the image return system is fixed, whereas the second mirror 16 of the image return system is movable.

Indeed, while the first mirror 15 is mounted on a fixed support, the second mirror 16 is mounted on a second-mirror support 19 that is rotatable about a second-mirror support axis of rotation 19.1.

The vision testing device 10 comprises a second-mirror support actuating device and a second-mirror support drive device (not shown) comprising at least one motor.

In particular, the second mirror 16 is movable between a deployed second-mirror position (visible in FIG. 2), occupied by the second mirror 16 when the vision testing device 10 occupies the first configuration, and a retracted second-mirror position (visible in FIG. 3), occupied by the second mirror 16 when the vision testing device 10 occupies the second configuration.

The second-mirror support 19 is adapted to switch the second mirror 16 from the deployed second-mirror position to the retracted second-mirror position, and vice versa.

The image return system further comprises a third mirror 12 arranged on the first optical path 30 between the image display device 70 and the second mirror 16 when the second mirror 16 is in the deployed second-mirror position, that is to say when the vision testing device 10 occupies the first configuration.

Furthermore, when the second mirror 16 is in the retracted second-mirror position, that is to say when the vision testing device 10 occupies the second configuration, the third mirror 12 is arranged on the second optical path 40.

The vision testing device 10 also comprises a second optical path prism 23 mounted in a removable way (and thus interchangeable) on a second optical path prism support (not referenced) and arranged on the second optical path 40 between the third mirror 12 and the observation position of the patient's eye 50, in the vicinity of the second optical path opening 21.

Similarly, the vision testing device 10 also comprises an additional second optical path prism 24 mounted in a removable way (and thus interchangeable) on an additional second optical path prism support (not referenced) and arranged on the additional second optical path between the third mirror 12 and the observation position of the patient's other eye 60, in the vicinity of the additional second optical path opening 22.

Thus, when the second mirror 16 occupies the retracted second-mirror position, that is to say when the vision testing device 10 occupies the second configuration, the third mirror 12 is arranged on the second optical path 40 between the image display device 70 and the second optical path prism 23, as well as on the additional second optical path between the image display device 70 and the additional second optical path prism 24.

The image return system is adapted to return the image or images displayed on the image display device 70, in particular by virtue of the specific arrangement of the first, second, and third mirrors (15, 16, 12).

When the vision testing device 10 occupies the first configuration, the third mirror 12 is intended to cooperate with the second mirror 16 occupying the deployed second-mirror position, and the second mirror 16 occupying the deployed second-mirror position is intended to cooperate with the first mirror 15. Furthermore, the second mirror 16 occupying the deployed second-mirror position obstructs the second optical path 40 as well as the additional second optical path.

As can be seen in FIG. 2, the first optical path 30 thus comprises:
a first portion of the first optical path 30.1 extending between the image display device 70 and the third mirror 12;
a second portion of the first optical path 30.2 extending between the third mirror 12 and the second mirror 16;
a third portion of the first optical path 30.3 extending between the second mirror 16 and the first mirror 15;
a fourth portion of the first optical path 30.4 extending between the first mirror 15 and the observation position of the patient's eye 50, passing through the housing position and the primary lens of the first optical path 17.

The additional first optical path further extends parallel to the first optical path 30, a fourth portion of the additional first optical path extending between the first mirror 15 and the observation position of the patient's other eye 60, passing through the additional housing position and the primary lens of the additional first optical path 18.

When the vision testing device 10 occupies the second configuration, the second mirror 16 occupies the retracted second-mirror position, and the third mirror is intended to return the images displayed on the image display device directly toward the observation position of the patient's eye 50. Moreover, when it occupies the retracted second-mirror position, the second mirror 16 clears the second optical path 40 as well as the additional second optical path, and both the first optical path 30 and the additional first optical path are interrupted.

Thus, as can be seen in FIG. 3, the second optical path 40 comprises:
a first portion of the second optical path 40.1 extending between the image display device 70 and the third mirror 12;
a second portion of the second optical path 40.2 extending between the third mirror 12 and the observation position of the patient's eye 50, passing through the second optical path prism 23.

The additional second optical path further extends parallel to the second optical path 40, a second portion of the additional second optical path extending between the third mirror 12 and the observation position of the patient's other eye 60, passing through the additional second optical path prism 24.

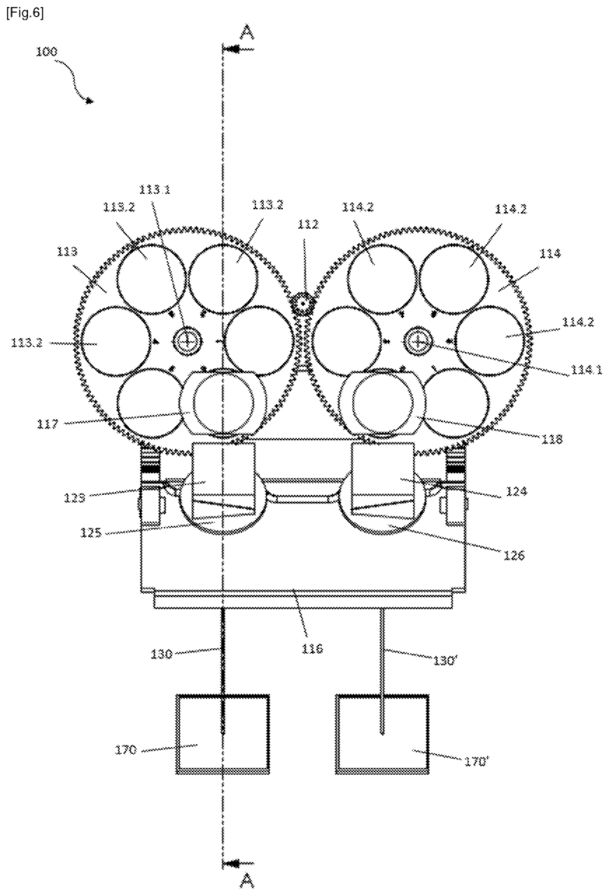

Like the vision testing device 10, the vision testing device 100 of FIGS. 6 to 8 makes it possible to test the overall vision of a patient, and in particular to detect visual defects affecting one or the other of the patient's eyes and/or to carry out a complete diagnosis of the patient's vision.

Certain elements common to the vision testing device 10 and to the vision testing device 100, already described above, will not be described again in detail below.

The vision testing device 100 comprises means (not shown) for defining an observation position of a patient's eye 150 and an observation position of the patient's other eye (not shown).

An image display device 170, remote from the vision testing device 100, makes it possible, for example, to display optotypes or other symbols observable by the patient's eye 150 from the observation position of the patient's eye 150.

Moreover, an additional image display device 170′ makes it possible, for example, to display optotypes or other symbols observable by the patient's other eye from the observation position of the patient's other eye.

The vision testing device 100 comprises a casing (not shown) intended to accommodate and protect at least some of the elements of the vision testing device 100, and both the observation position of the patient's eye 150 and the observation position of the patient's other eye are located outside said casing.

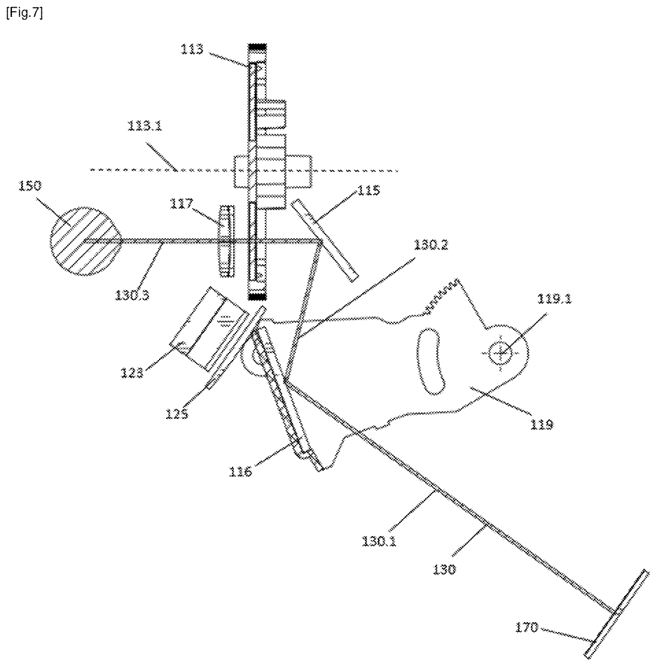

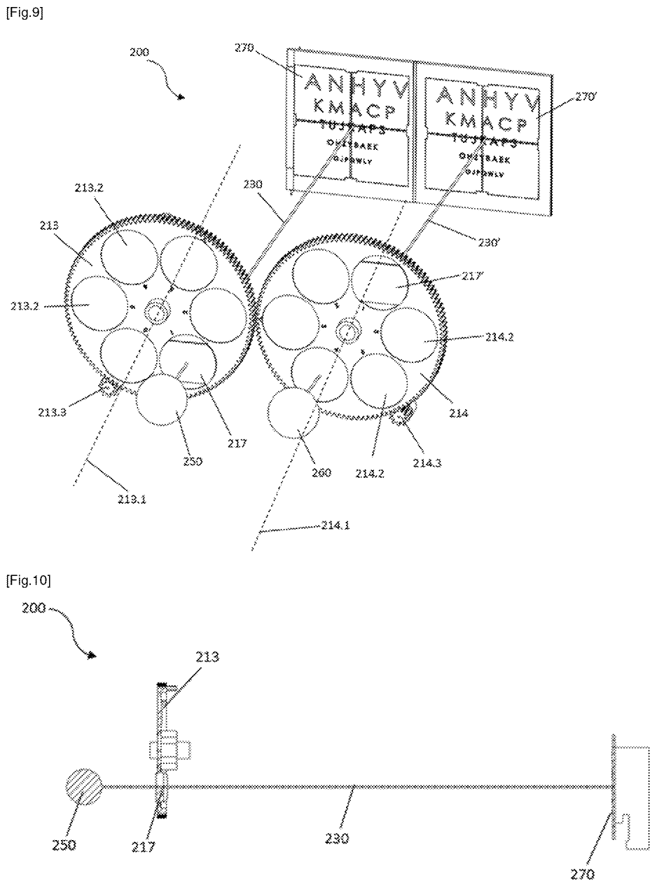

The vision testing device 100 is designed to be adapted to occupy:
a first configuration, or distance vision configuration, illustrated in FIG. 7, in which a first optical path 130, or distance vision optical path, is defined between the observation position of the patient's eye 150 and the image display device 170, and in which an additional first optical path 130′, or additional distance vision optical path, is defined between the observation position of the patient's other eye and the additional image display device 170′;
a second configuration, or near vision configuration, illustrated in FIG. 10, in which a second optical path 140, or near vision optical path, is defined between the observation position of the patient's eye 150 and the image display device 170, and in which an additional second optical path, or additional near vision optical path (not shown in the figures), is defined between the observation position of the patient's other eye and the additional image display device 170′.

In order to define, respectively, the first optical path 130, the additional first optical path 130′, the second optical path 140, and the additional second optical path, the casing of the vision testing device 100 has different first optical path openings and different second optical path openings, not shown in the figures.

The vision testing device 100 also comprises an image return system adapted, on the one hand, to return the images displayed on the image display device 170 respectively toward the observation position of the patient's eye 150 and the observation position of the patient's other eye, and, on the other hand, to return the images displayed on the additional image display device 170′ respectively toward the observation position of the patient's eye 150 and the observation position of the patient's other eye.

More particularly:
When the vision testing device 100 occupies the first configuration, the images returned from the image display device 170 travel along the first optical path 130 to reach the patient's eye 150, and the images returned from the additional image display device 170′ travel along the additional first optical path 130′ to reach the patient's other eye;
When the vision testing device 100 occupies the second configuration, the images returned from the image display device 170 travel along the second optical path 140 to reach the patient's eye 150, and the images returned from the additional image display device 170′ travel along the additional second optical path to reach the patient's other eye.

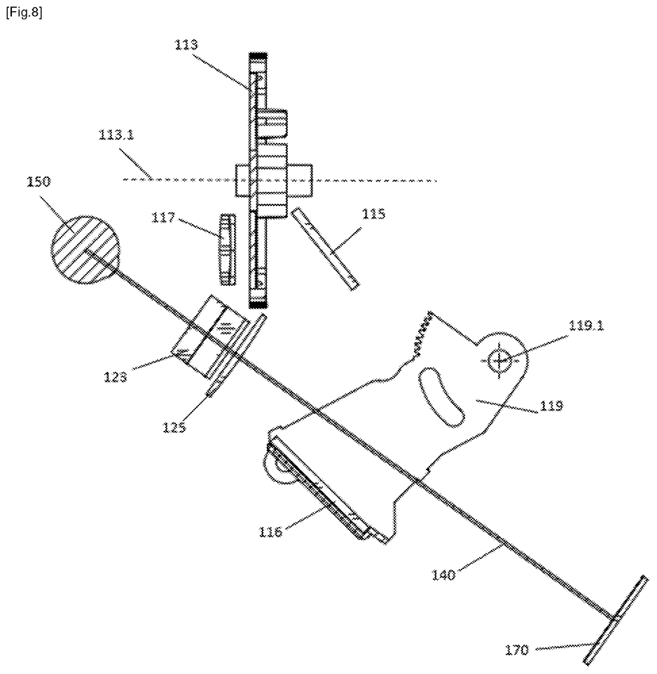

The image return system comprises a first mirror 115 (visible in FIGS. 7 and 8) and a second mirror 116.

The vision testing device 100 also comprises a primary lens of the first optical path 117 and a primary lens of the additional first optical path 118, which are arranged to be respectively positioned on the first optical path 130 facing the observation position of the patient's eye 150, and on the additional first optical path 130′ facing the observation position of the patient's other eye.

The vision testing device 100 further comprises a movable member 113 having six movable member housings 113.2 intended to alternately occupy, on the first optical path 130, a housing position by rotation of the movable member 113 about a movable member axis of rotation 113.1 that is substantially coplanar with and parallel to a direction of a portion of the first optical path extending between the observation position of the patient's eye 150 and the housing position.

The six movable member housings 113.2 are identical to one another, each being respectively adapted to receive an optical element (not shown), such as for example a lens.

The vision testing device 100 also comprises an additional movable member 114 having six additional movable member housings 114.2 intended to alternately occupy, on the additional first optical path 130′, an additional housing position by rotation of the additional movable member 114 about an additional movable member axis of rotation 114.1 that is substantially coplanar with and parallel to a direction of a portion of the additional first optical path (not visible in the figures) extending between the observation position of the patient's other eye and the additional housing position.

The primary lens of the first optical path 117 is arranged on the first optical path 130 between the observation position of the patient's eye 150 and the housing position, and the primary lens of the additional first optical path 118 is arranged on the additional first optical path 130′ between the observation position of the patient's other eye and the additional housing position.

The six additional movable member housings 114.2 are identical to one another and are also identical to the six movable member housings 113.2, each of the six additional movable member housings 114.2 being respectively adapted to receive an optical element, such as for example a lens.

The six movable member housings 113.2 are shown in FIG. 6 without optical elements.

The six movable member housings 114.2 are shown in FIG. 6 without optical elements.

The image return system is partially movable so as to switch the vision testing device 100 from the first configuration to the second configuration, and vice versa.

The first mirror 115 of the image return system is fixed, whereas the second mirror 116 of the image return system is movable.

Indeed, while the first mirror 115 is mounted on a fixed first mirror support, the second mirror 116 is mounted on a second-mirror support 119 that is rotatable about a second-mirror support axis of rotation 119.1.

The vision testing device 100 comprises a second-mirror support actuating device and a second-mirror support drive device (not shown) comprising at least one motor.

In particular, the second mirror 116 is movable between a deployed second-mirror position (visible in FIG. 7) occupied by the second mirror 116 when the vision testing device 100 occupies the first configuration, and a retracted second-mirror position (visible in FIG. 8) occupied by the second mirror 116 when the vision testing device 100 occupies the second configuration.

The second-mirror support 119 is therefore adapted to switch the second mirror 116 from the deployed second-mirror position to the retracted second-mirror position, and vice versa.

The vision testing device 100 also comprises a second optical path prism 123 mounted in a removable way (and therefore interchangeable) on a second optical path prism support (not referenced) and arranged on the second optical path 140. The vision testing device 100 also comprises a second optical path lens 125 mounted in a removable way on a second optical path lens support (not shown), arranged on the second optical path 140 and associated with the second optical path prism 123.

Similarly, the vision testing device 100 also comprises an additional second optical path prism 124 mounted in a removable way (and thus interchangeable) on an additional second optical path prism support (not referenced) and arranged on the additional second optical path. The vision testing device 100 also comprises an additional second optical path lens 126 mounted in a removable way on an additional second optical path lens support (not shown), arranged on the additional second optical path and associated with the additional second optical path prism 124.

When the vision testing device 100 occupies the first configuration, the second mirror 116 occupying the deployed second-mirror position, is intended to cooperate with the first mirror 115. Furthermore, the second mirror 116, occupying the deployed second-mirror position, obstructs the second optical path 140 as well as the additional second optical path.

As can be seen in FIG. 7, the first optical path 130 thus comprises:
a first portion of the first optical path 130.1 extending between the image display device 170 and the second mirror 116;
a second portion of the first optical path 130.2 extending between the second mirror 116 and the first mirror 115;
a third portion of the first optical path 130.3 extends between the first mirror 15 and the observation position of the patient's eye 150, passing through the housing position and the primary lens of the first optical path 117.

The additional first optical path 130′ further extends parallel to the first optical path 130 starting from the additional display device 170′, a third portion of the additional first optical path extending between the first mirror 115 and the observation position of the patient's other eye, passing through the additional housing position and the primary lens of the additional first optical path 118.

When the vision testing device 100 is in the second configuration, the second mirror 116 occupies the retracted second-mirror position, the images displayed on the image display device 170 being directly reflected back toward the observation position of the patient's eye 150 while passing through the second optical path lens 125 and the second optical path prism 123. Moreover, when it occupies the retracted second-mirror position, the second mirror 116 clears the second optical path 140 as well as the second additional optical path, and both the first optical path 130 and the additional first optical path 130′ are interrupted.

Thus, as can be seen in FIG. 8, the second optical path 140 extends directly between the image display device 170 and the observation position of the patient's eye 150, passing through the second optical path lens 125 and the second optical path prism 123.

The additional second optical path further extends parallel to the second optical path 140, directly between the additional image display device 170′ and the observation position of the patient's other eye, passing through the additional second optical path lens 126 and the additional second optical path prism 124.

The movable member 113 and the additional movable member 114 are configured to be rotatable in a synchronized manner. In other words, a rotational movement of the movable member 113 (respectively of the additional movable member 114) causes, in a synchronized manner, a rotational movement of the additional movable member 114 (respectively the movable member 113).

Thus, when the movable member 113 rotates at a given speed, the movable member 114 also rotates at the same speed.

The synchronized rotational mobility of the movable member 113 and the additional movable member 114 is obtained by means of a drive mechanism, a first drive mechanism part being provided on the movable member 113, a second drive mechanism part being provided on the additional movable member 114, and a third drive mechanism part comprising a pinion 112 rotatable in engagement with both the first part of the drive mechanism and the second part of the drive mechanism.

More specifically, the first drive mechanism part comprises a first set of teeth arranged on the periphery, and in particular on the circumference, of the movable member 113, the second drive mechanism part comprises a second set of teeth arranged on the periphery, and in particular on the circumference, of the additional movable member 114; and the pinion 112 comprises a third set of teeth complementary to both the first set of teeth and the second set of teeth.

More specifically, the pinion 112 has a thin disc shape and comprises, at its periphery, in particular on its circumference, the third set of teeth.

Thanks to this arrangement, and in particular to the rotating pinion 112, the direction of rotation of the additional movable member 114 is identical to the direction of rotation of the movable member 113. Moreover, the additional movable member 114 is strictly identical to the movable member 113.

Finally, the vision testing device 100 comprises a pinion actuating device and a pinion drive device (not shown) comprising at least one motor. Thus, the pinion 112 is motorized.

The patient vision testing device 200, shown in FIGS. 9 and 10, is a simpler and less costly version than the previously described vision testing devices 10 and 100, and can thus advantageously be used for large-scale screening operations, involving, for example, a large part of a given population.

The vision testing device 200 is in particular devoid of a primary optical element of the first optical path.

Certain elements common to the vision testing devices 10, 100, and 200, already described above, will not be described again in detail below.

The vision testing device 200 is intended to occupy a first configuration in which a first optical path 230, which may be a near vision, distance vision, or intermediate vision optical path, is provided between an observation position of a patient's eye 250 and a remote image display device 270.

The vision testing device 200 comprises a movable member 213 having six movable member housings 213.2 intended to alternately occupy, on the first optical path 230, a housing position by rotation of the movable member 213 about a movable member axis of rotation 213.1 substantially coplanar with and parallel to a direction of a portion of the first optical path 230 extending between the observation position of the patient's eye 250 and the housing position.

Each of the six movable member housings 213.2 permanently integrates a secondary optical element of the first optical path, for example, a secondary lens of the first optical path or a prism of the first optical path 217.

Moreover, an additional first optical path 230′, which may be a near, distance vision, or intermediate vision optical path, is also provided between an observation position of the patient's other eye 260 and a remote additional image display device 270′, and the vision testing device further comprises an additional movable member 214 having six additional movable member housings 214.2 intended to occupy alternately, on the additional first optical path 230′, an additional housing position by rotation of the additional movable member 214 about an additional movable member axis of rotation 214.1 that is substantially coplanar with and parallel to a direction of a portion of the additional first optical path 230′ extending between the observation position of the patient's other eye 260 and the additional housing position.

Each of the six additional movable members 214.2 permanently integrates a secondary optical element of the first optical path, for example, a secondary lens of the additional first optical path or a prism of the additional first optical path 217′.

It is understood that, depending on the nature and/or properties of the secondary optical elements of the first optical path provided on the vision testing device 200, the latter can allow testing, while occupying a single and same configuration, near vision, distance vision, or intermediate vision, simply by rotation of the movable member 213 and/or the additional movable member 214.

The movable member 213 and the additional movable member 214 of the vision testing device are configured to be rotatable independently of one another. In other words, the respective movements of the movable member 213 and the additional movable member are desynchronized, and the movable member 213 may move while the additional movable member remains stationary, or vice versa.

Moreover, the respective rotational speeds of the movable member and of the additional movable member are not necessarily identical.

The movable member 213 has its own drive mechanism, in particular consisting of a motorized or non-motorized pinion 213.3.

Similarly, the additional movable member 214 also has its own drive mechanism consisting of a motorized or non-motorized pinion 214.3.

The disclosure is obviously not limited to the three embodiments shown in the figures and described above, and encompasses all variants conceivable by those skilled in the art. In particular, the various non-incompatible elements described above can be combined in any number of variants.

1. A patient vision testing device designed to occupy at least one first configuration in which a first optical path is provided between an observation position of one eye of the patient and an image display device, the testing device comprising a movable member having at least two movable member housings intended to alternately occupy a housing position on the first optical path by rotation of the movable member about a rotation axis of a movable member substantially coplanar and parallel to a direction of a portion of the first optical path extending between the observation position of the patient's eye and the housing position.

2. The testing device according to claim 1, further comprising a primary optical element of the first optical path disposed on the first optical path.

3. The testing device according to claim 1, wherein:
each of said at least two movable member housings permanently incorporates a secondary first-path optical element, or wherein:
each of said at least two movable member housings is capable of temporarily housing a secondary first-path optical element.

4. The testing device according to claim 2, wherein the primary first-path optical element is a primary lens of the first optical path.

5. The testing device according to claim 3, wherein the additional first optical path element is a secondary lens of the first optical path or a first-path prism.

6. The testing device according to claim 1, further configured to occupy a second position wherein a second optical path is provided between the observation position of the patient's eye and the image display device, the testing device further comprising an image return system including a first mirror and a second mirror.

7. The testing device according to claim 6, wherein the image feedback system is at least partially movable to allow the vision testing device to be moved from the first configuration to the second configuration and vice versa.

8. The testing device according to claim 6, wherein the second mirror is movable between a deployed second-mirror position occupied by the second mirror when the vision testing device is in the first configuration, and a retracted second-mirror position occupied by the second mirror when the vision testing device is in the second configuration.

9. The testing device according to claim 6, further comprising at least one second-path optical element disposed on the second optical path.

10. The testing device according to claim 9, wherein at least one second-path optical element comprises a second-path prism and/or a second-path lens.

11. The testing device according to claim 6, wherein the image return system comprises a third mirror.

12. The testing device according to claim 1, wherein, when the vision testing device is in the first configuration, an additional first optical path is provided between an observation position of the patient's other eye and the image display device, the vision testing device comprising an additional movable member having at least two additional movable member housings intended to alternately occupy an additional housing position on the additional first optical path, by rotation of the additional movable member about an additional movable member rotation axis substantially coplanar and parallel to a direction of a portion of the additional first optical path extending between the observation position of the patient's other eye and the additional housing position.

13. The testing device according to claim 12, wherein the movable member and the additional movable member are configured to rotate in a synchronized manner.

14. The testing device according to claim 12, comprising a drive mechanism capable of simultaneously rotating the movable member and the additional movable member.

15. The testing device according to claim 14, wherein the drive mechanism comprises a first drive mechanism part provided on the movable member, a second drive mechanism part provided on the additional movable member, and a third drive mechanism part comprising a rotatable pinion.

16. A method of using the testing device according to claim 1.
